(19) United States
(12) Patent Application Publication
(10) Pub. No.: US 2020/0029944 A1
(43) Pub. Date: Jan. 30, 2020

(54) ENDOSCOPIC BIOPSY ASSEMBLY

(71) Applicant: William Lunt, Colorado Springs, CO (US)

(72) Inventor: William Lunt, Colorado Springs, CO (US)

(21) Appl. No.: 16/045,051

(22) Filed: Jul. 25, 2018

(51) Int. Cl. A61B 10/04; A61B 10/06; A61B 10/02

(52) U.S. Cl. CPC A61B 10/04; A61B 10/06; A61B 17/320016; A61B 2010/0225; A61B 10/0283

(57) ABSTRACT

An endoscopic biopsy assembly includes an endoscope that has a working channel and a suction device. A biopsy unit is provided and the biopsy unit is insertable into the working channel in the endoscope for obtaining a tissue biopsy. The biopsy unit is in fluid communication with the suction device such that the suction device urges air inwardly through the biopsy device and into a tissue trap. The biopsy unit is urgeable into a cutting position for obtaining the tissue biopsy. The suction device suctionally urges the tissue biopsy out of the biopsy unit and into the tissue trap thereby facilitating the tissue biopsy to be acquired without removing the biopsy unit from the endoscope. Moreover, the biopsy unit is repeatedly urgeable into the cutting position for obtaining and thusly acquiring multiple tissue biopsies without removing the biopsy unit from the endoscope.

### CROSS-REFERENCE TO RELATED APPLICATIONS

### Statement Regarding Federally Sponsored Research or Development

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

(1) Field of the Invention

(2) Description of Related Art Including Information Disclosed Under 37 CFR 1.97 and 1.98.

The disclosure and prior art relates to biopsy devices and more particularly pertains to a new biopsy device for obtaining multiple biopsies without removing a reinserting a biopsy unit in an endoscope.

### BRIEF SUMMARY OF THE INVENTION

An embodiment of the disclosure meets the needs presented above by generally comprising an endoscope that has a working channel and a suction device. A biopsy unit is provided and the biopsy unit is insertable into the working channel in the endoscope for obtaining a tissue biopsy. The biopsy unit is in fluid communication with the suction device such that the suction device urges air inwardly through the biopsy device and into a tissue trap. The biopsy unit is urgeable into a cutting position for obtaining the tissue biopsy. The suction device suctionally urges the tissue biopsy out of the biopsy unit and into the tissue trap thereby facilitating the tissue biopsy to be acquired without removing the biopsy unit from the endoscope. Moreover, the biopsy unit is repeatedly urgeable into the cutting position for obtaining and thusly acquiring multiple tissue biopsies without removing the biopsy unit from the endoscope.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a biopsy unit endoscopic biopsy assembly according to an embodiment of the disclosure.

FIG. 2 is a perspective view of an embodiment of the disclosure.

FIG. 3 is a front phantom view of an embodiment of the disclosure.

FIG. 4 is a cross sectional view taken along line 4-4 of FIG. 3 of an embodiment of the disclosure.

FIG. 5 is a perspective phantom view of a pair of blades an embodiment of the disclosure in an open position.

FIG. 6 is a perspective phantom view of a pair of blades an embodiment of the disclosure in a cutting position.

### DETAILED DESCRIPTION OF THE INVENTION

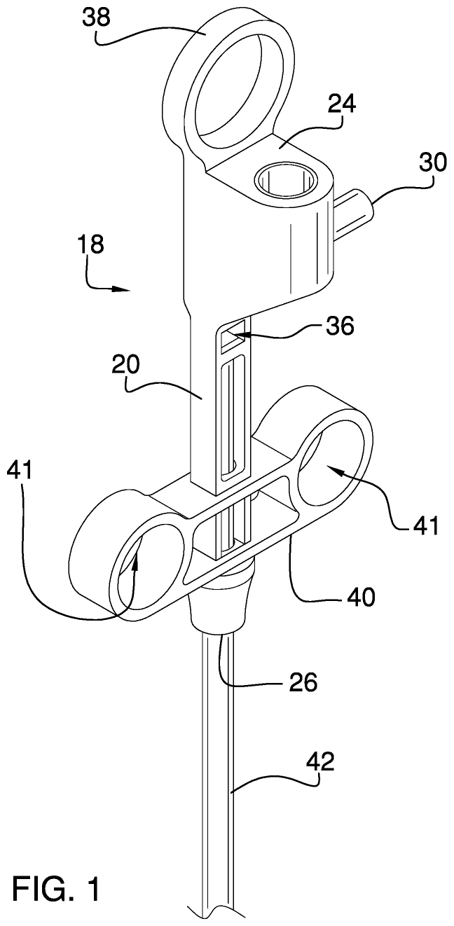

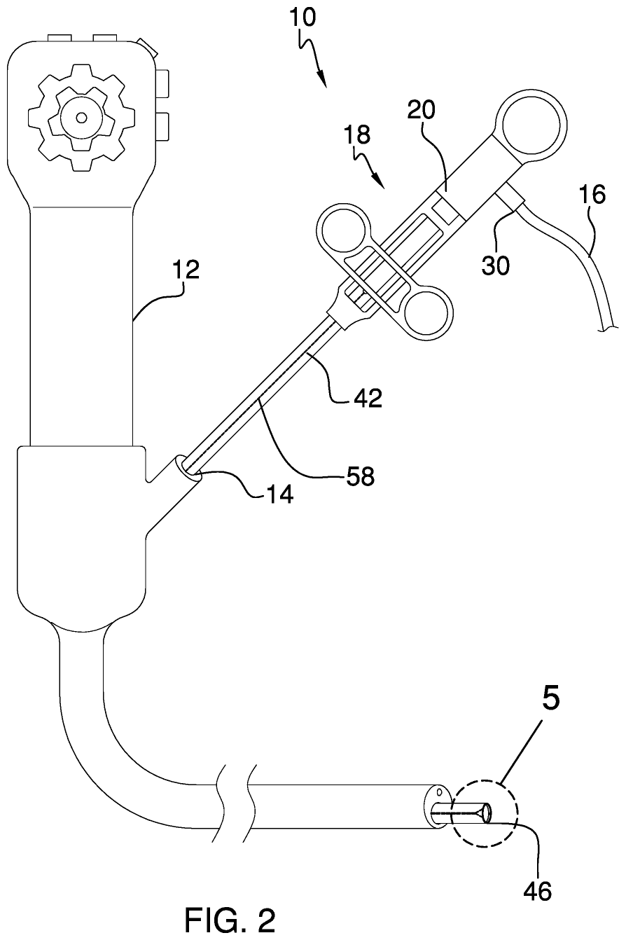

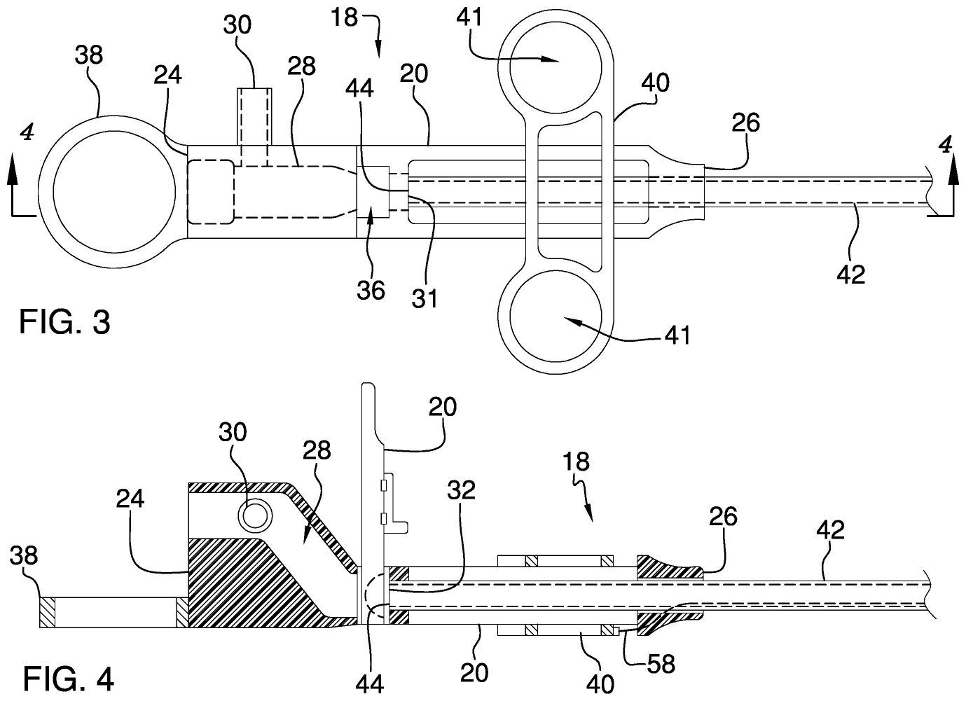

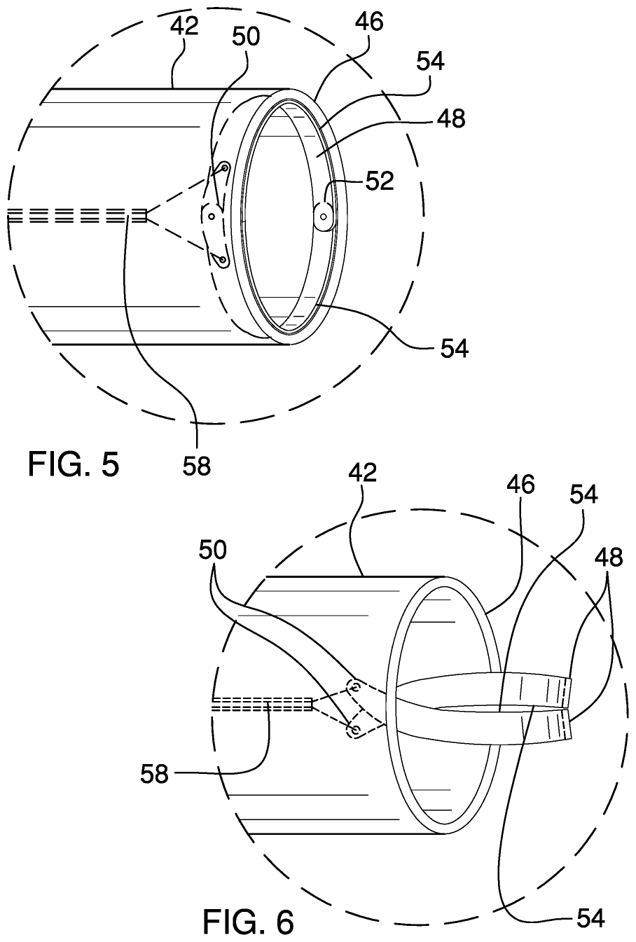

With reference now to the drawings, and in particular to FIGS. 1 through 6 thereof, a new biopsy device embodying the principles and concepts of an embodiment of the disclosure and generally designated by the reference numeral 10 will be described.

As best illustrated in FIGS. 1 through 6, the endoscopic biopsy assembly 10 generally comprises an endoscope 12 that has a working channel 14. The endoscope 12 may be an endoscope 12 employed during gastroenterological procedures or the like. A suction device 16 is included and the suction device 16 may a suction device 16 of any conventional design that is approved for use in an operating room of a hospital.

A biopsy unit 18 is insertable into the working channel 14 in the endoscope 12 for obtaining a tissue biopsy. Additionally, the biopsy unit 18 is in fluid communication with the suction device 16 such that the suction device 16 urges air inwardly through the biopsy device and into a tissue trap 20. The biopsy unit 18 is urgeable into a cutting position for obtaining the tissue biopsy, such as a sample of a large intestine, a stomach or other digestive organs commonly biopsied for diagnostic purposes. The suction device 16 suctionally urges the tissue biopsy out of the biopsy unit 18 and into the tissue trap 20. In this way the tissue biopsy may be acquired without removing the biopsy unit 18 from the endoscope 12. Moreover, the biopsy unit 18 is repeatedly urgeable into the cutting position for obtaining and thusly acquiring multiple tissue biopsies without removing the biopsy unit 18 from the endoscope 12. In this way the biopsy unit 18 in conjunction with the suction device 16 improves the number of biopsies that can be acquired and the speed with which the biopsies can be acquired.

The biopsy unit 18 comprises a handle 22 that has a first end 24 and a second end 26, and the handle 22 is elongated between the first 22 and second 26 ends. The handle 22 has a suction conduit 28 integrated therein, and the suction conduit 28 has an input 30 and an output 32. The input 30 is fluidly coupled to the suction device 16 such that the suction device 16 urges air inwardly through the output 32. Moreover, the first end 24 has a suction port 34 extending into the suction conduit 28 such that the suction port 34 is in fluid communication with the suction device 16. The suction device 16 urges air inwardly through the suction port 34 when the suction port 34 is unobstructed thereby inhibiting air from being urged inwardly through the output 32. Alternatively, the suction device 16 urges air through the output 32 when the suction port 34 is obstructed by a user's thumb.

The handle 22 has a tissue trap aperture 36 extending therethrough and the tissue trap aperture 36 intersects the suction conduit 28. The tissue trap 20 is slidable into the tissue trap aperture 36. Moreover, the tissue trap 20 is comprised of an air permeable material such that the suction device 16 urges air through the tissue trap 20. The tissue trap 20 may include a slide for slidably inserting in the tissue trap 20 and a screen positioned in the slide for capturing the tissue.

A thumb ring 38 extends upwardly from the first end 24 of the handle 22 and the user's thumb is extended through the thumb ring 38. The thumb ring 38 is offset from the suction port 34 in the first end 24 such that the user can selectively position their thumb over the suction port 34 when the thumb is extended through the thumb ring 38. A grip 40 is included and grip 40 is slidably coupled around the handle 22. The grip 40 has a pair of finger apertures 41 therein for having a respective one of the user's fingers extended therethrough. The grip 40 is urgeable into a cutting position having the grip 40 being slid upwardly along the handle 22 toward the thumb ring 38. Alternatively, the grip 40 is urgeable into an opening position having the grip 40 being slid downwardly along the handle 22 away from the thumb ring 38.

A suction tube 42 is included that has a primary end 44 and a secondary end 46. The primary end 44 is fluidly coupled to the output 32 of the suction conduit 28 having the suction tube 42 extending outwardly through the second end 26 of the handle 22. The suction tube 42 is comprised of a flexible material and the secondary end 46 of the suction tube 42 is insertable into the working channel 14 of the endoscope 12. The suction tube 42 has a length that is at least great enough to extend along a full length of the endoscope 12 thereby facilitating the secondary end 46 of the suction tube 42 to extend outwardly from a distal end of the endoscope 12.

A pair of blades 48 is included and each of the blades 48 has a first end 50, a second end 52 and a cutting edge 54 extending therebetween. The cutting edge 54 of each of the blades 48 is concavely arcuate between the first 50 and second 52 ends of the blades 48 such that each of the blades 48 has a semi-circular shape. The first 50 and second 52 ends of each of the blades 48 are pivotally coupled to an inside surface 56 of the suction tube 42. The blades 48 are oriented to curve away from each other along the inside surface 56 of the suction tube 42.

Each of the blades 48 is positionable in an open position having the cutting edge 54 of each of the blades 48 being aligned with and being collinear with the second end 26 of the suction tube 42. Additionally, the blades 48 are positioned to extend around an entire inside diameter of the suction tube 42 when the blades 48 are in the open position. Each of the blades 48 is positionable in a cutting position having such cutting edge 54 of each of the blades 48 being urged outwardly from the second end 26 of the suction tube 42. Additionally, the cutting edge 54 on each of the blades 48 and engages each other for cutting the tissue biopsy.

A wire 58 is coupled between the grip 40 and the first end 24 of each of the blades 48. The wire 58 urges each of the blades 48 into the open position when the grip 40 is urged into the opening position. Alternatively, the wire 58 urges each of the blades 48 into the cutting position when the grip 40 is urged into the cutting position.

In use, the suction tube 42 is inserted into the endoscope 12 when the endoscope 12 has been located for the biopsy. The suction device 16 is coupled to the input 30 of the suction port 34, the fingers are inserted through the finger apertures 41 in the grip 40 and the thumb is inserted into the thumb ring 38. The grip 40 is urged toward the thumb ring 38 to close the blades 48 and thereby acquire the tissue biopsy. The user's thumb is positioned over the suction port 34 thereby facilitating the suction device 16 to suck the tissue biopsy through the suction tube 42 to be captured in the tissue trap 20. The tissue trap 20 is removed, a fresh tissue trap 20 is inserted into the handle 22 and the process of acquiring a tissue biopsy is repeated. Thus, multiple tissue biopsies can be acquired without removing the biopsy unit 18 from the endoscope 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An endoscopic biopsy assembly being configured to acquire multiple biopsies during an endoscopic procedure, said assembly comprising:
an endoscope having a working channel;
a suction device having tissue trap; and
a biopsy unit being insertable into said working channel in said endoscope for obtaining a tissue biopsy, said biopsy unit being in fluid communication with said suction device such that said suction device urges air inwardly through said biopsy device and into said tissue trap, said biopsy unit being urgeable into a cutting position for obtaining the tissue biopsy, said suction device suctionally urging the tissue biopsy out of said biopsy unit and into said tissue trap thereby facilitating the tissue biopsy to be acquired without removing said biopsy unit from said endoscope, said biopsy unit being repeatedly urgeable into said cutting position for obtaining and thusly acquiring multiple tissue biopsies without removing said biopsy unit from said endoscope.

2. The assembly according to claim 1, wherein:
said biopsy unit comprises a handle having a first end and a second end, said handle being elongated between said first and second ends, said handle having a suction conduit being integrated therein, said suction conduit having an input and an output, said input being fluidly coupled to said suction unit such that said suction unit urges air inwardly through said output;
said first end having a suction port extending into said suction conduit having said suction port being in fluid communication with said suction unit, said suction unit urging air inwardly through said suction port when said suction port is unobstructed thereby inhibiting air from being urged inwardly through said output, said suction unit urging air through said output when said suction port is obstructed by a user's thumb;
said handle having a tissue trap aperture extending therethrough and intersecting said suction conduit; and
said tissue trap being slidable into said tissue trap aperture, said tissue trap being comprised of an air permeable material such that said suction unit urges air through said tissue trap.

3. The assembly according to claim 2, further comprising:
a thumb ring extending upwardly from said first end of said handle wherein said thumb ring is configured to have the user's thumb extended therethrough, said thumb ring being offset from said suction port in said first end; and
a grip being slidably coupled around said handle, said grip having a pair of finger apertures therein wherein each of said finger apertures is configured to have a respective one of the user's fingers extended therethrough, said grip being urgeable into a cutting position having said grip being slid upwardly along said handle toward said thumb ring, said grip being urgeable into an opening position having said grip being slid downwardly along said handle away from said thumb ring.

4. The assembly according to claim 3, further comprising a suction tube having a primary end and a secondary end, said primary end being fluidly coupled to said output of said suction conduit having said suction tube extending outwardly through said second end of said handle, said suction tube being comprised of a flexible material, said secondary end of said suction tube being insertable into said working channel of said endoscope.

5. The assembly according to claim 4, further comprising a pair of blades, each of said blades having a first end, a second end and a cutting edge extending therebetween, said cutting edge of each of said blades being concavely arcuate between said first and second ends of said blades such that each of said blades has a semi-circular shape, said first and second end of each of said blades being pivotally coupled to an inside surface of said suction tube.

6. The assembly according to claim 5, wherein each of said blades is positionable in an open position having said cutting edge of each of said blades being aligned with and being collinear with said second end of said suction tube.

7. The assembly according to claim 6, wherein each of said blades is positionable in a cutting position having such cutting edge of each of said blades being urged outwardly from said second end of said suction tube and engaging each other wherein said cutting edge of each of said blades is configured to cut the tissue biopsy.

8. The assembly according to claim 5, further comprising a wire being coupled between said grip and each of said blades, said wire urging each of said blades into an open position when said grip is urged into said opening position, said wire urging each of said blades into a cutting position when said grip is urged into said cutting position.

9. An endoscopic biopsy assembly being configured to acquire multiple biopsies during an endoscopic procedure, said assembly comprising:
an endoscope having a working channel;
a suction device having tissue trap; and
a biopsy unit being insertable into said working channel in said endoscope for obtaining a tissue biopsy, said biopsy unit being in fluid communication with said suction device such that said suction device urges air inwardly through said biopsy device and into said tissue trap, said biopsy unit being urgeable into a cutting position for obtaining the tissue biopsy, said suction device suctionally urging the tissue biopsy out of said biopsy unit and into said tissue trap thereby facilitating the tissue biopsy to be acquired without removing said biopsy unit from said endoscope, said biopsy unit being repeatedly urgeable into said cutting position for obtaining and thusly acquiring multiple tissue biopsies without removing said biopsy unit from said endoscope, said biopsy unit comprising:
a handle having a first end and a second end, said handle being elongated between said first and second ends, said handle having a suction conduit being integrated therein, said suction conduit having an input and an output, said input being fluidly coupled to said suction unit such that said suction unit urges air inwardly through said output, said first end having a suction port extending into said suction conduit having said suction port being in fluid communication with said suction unit, said suction unit urging air inwardly through said suction port when said suction port is unobstructed thereby inhibiting air from being urged inwardly through said output, said suction unit urging air through said output when said suction port is obstructed by a user's thumb, said handle having a tissue trap aperture extending therethrough and intersecting said suction conduit;
said tissue trap being slidable into said tissue trap aperture, said tissue trap being comprised of an air permeable material such that said suction unit urges air through said tissue trap;
a thumb ring extending upwardly from said first end of said handle wherein said thumb ring is configured to have the user's thumb extended therethrough, said thumb ring being offset from said suction port in said first end;
a grip being slidably coupled around said handle, said grip having a pair of finger apertures therein wherein each of said finger apertures is configured to have a respective one of the user's fingers extended therethrough, said grip being urgeable into a cutting position having said grip being slid upwardly along said handle toward said thumb ring, said grip being urgeable into an opening position having said grip being slid downwardly along said handle away from said thumb ring;
a suction tube having a primary end and a secondary end, said primary end being fluidly coupled to said output of said suction conduit having said suction tube extending outwardly through said second end of said handle, said suction tube being comprised of a flexible material, said secondary end of said suction tube being insertable into said working channel of said endoscope;
a pair of blades, each of said blades having a first end, a second end and a cutting edge extending therebetween, said cutting edge of each of said blades being concavely arcuate between said first and second ends of said blades such that each of said blades has a semi-circular shape, said first and second end of each of said blades being pivotally coupled to an inside surface of said suction tube, each of said blades being positionable in an open position having said cutting edge of each of said blades being aligned with and being collinear with said second end of said suction tube, each of said blades being positionable in a cutting position having such cutting edge of each of said blades being urged outwardly from said second end of said suction tube and engaging each other wherein said cutting edge of each of said blades is configured to cut the tissue biopsy; and
a wire being coupled between said grip and each of said blades, said wire urging each of said blades into said open position when said grip is urged into said opening position, said wire urging each of said blades into said cutting position when said grip is urged into said cutting position.
